(19) United States
(12) Patent Application Publication
(10) Pub. No.: US 2016/0206378 A1
(43) Pub. Date: Jul. 21, 2016

(54) PRE-OPERATIVE JOINT DIAGNOSTICS

(71) Applicant: Corin Limited, Gloucestershire (GB)

(72) Inventors: Magnus Flett, Gloucestershire (GB); Stefano Alfonsi, Gloucestershire (GB)

(21) Appl. No.: 14/995,220

(22) Filed: Jan. 14, 2016

(30) Foreign Application Priority Data
Jan. 15, 2015 (GB) 1500650.5

(51) Int. Cl. A61B 34/10; A61B 5/00; A61B 5/11

(52) U.S. Cl. CPC A61B 34/10; A61B 5/1127; A61B 5/4528; A61B 5/7264; A61B 5/6801; A61B 5/0013; A61B 2560/0475; A61B 2562/0219; A61B 2034/107; A61B 2090/374

(57) ABSTRACT

A method of pre-operatively determining the suitability of a joint for a surgical corrective procedure, the method comprising the steps of: determining a patient's joint orientation in a plurality of situations; determining a patient kinematic range of the patient joint based on the said joint orientations; comparing the determined patient kinematic range to a preferred kinematic range; and determining that the joint is suitable for a first surgical corrective procedure if the patient kinematic range falls within the preferred kinematic range, else determining that the joint is suitable for a second surgical corrective procedure which is different from the said first surgical corrective procedure. A device for the determination of the patient kinematic range is also provided.

### RELATED APPLICATIONS

The present application claims the benefit under 35 U.S.C. §119(a)-(d) of British Patent

Application No. 1500650.5 filed Jan. 15, 2015, the disclosure of which is incorporated herein by reference.

### TECHNICAL FIELD

The present invention relates to a joint-orientation monitoring system for the creation of patient kinematic range data, particularly, but not necessarily exclusively, for the selection of a surgical procedure. The invention also relates to a method of pre-operatively determining the suitability of a joint for said surgical procedure.

### BACKGROUND

Old age, wear, and disease all contribute to the deterioration of skeletal joints. As the average age of the general population grows, it is becoming ever more necessary to perform surgery on various joints, such as the hip joint or knee joint, for either maintenance or replacement purposes.

Such surgery can be very invasive, and thus traumatic for the patient. For instance, in order to perform a hip arthroplasty, it is necessary to create a large incision in the patient and then forcibly dislocate the femur from the acetabulum. The use of such a drastic procedure is therefore carefully limited to only those who will benefit to the greatest extent.

Given the limited biomedical lifespan of artificial implants, multiple surgical procedures may be required to maintain operable functionality of the joint, especially in view of the increased longevity of humans. The longevity of these replacement joints is further limited, in some cases, by the particular anomalies and anatomical quirks of the patient.

Design and implantation of artificial joints in the past has had to be based upon a generic patient biometry, in order to be focussed on providing the greatest benefit to the greatest number of people. As technology has progressed, it has become possible to make tailored artificial joints that are bespoke to each individual patient, and this has led to increased life-span of joint replacements and a higher overall success rate.

However, the method of surgical implantation has remained generic in comparison. It is commonplace for patients to be submitted for medical imaging to determine the anatomy of the patient's joint and then to fit the artificial joint in the position deemed most suitable based on this information. Whilst this is acceptable for many patients, it does not result in high success rates in others.

There are many possible reasons why generic surgery is not suitable in all situations, but one example is that the anatomy of patients' joints responds to movement in different ways. Taking the hip joint as an example, whilst standing still the front of the pelvis may drop slightly and the back rise in what is known as an anterior pelvic tilt. Similarly, whilst seated the reverse may be true and the pelvis may have a posterior pelvic tilt. It is equally plausible that during some activities the pelvis may be tilted laterally.

The existence and degree of pelvic tilt during static and dynamic activities may vary between patients, which leads to replacement joints wearing at different speeds and in contrasting places, as forces are transmitted through various portions of the joint. This can lead to failures as, for instance, the side of the acetabular cup portion of the replacement hip joint is forced to bear more load than it is designed for, due to abnormal pelvic tilt.

Recent developments in the field have led to the creation of techniques which consist of extensive pre-operative investigation of the patient in order to provide a more bespoke surgical method. These methods allow a much more tailored approach to the surgery, whereby the particular anatomical differences between patients are taken into account before deciding on specific alterations to the generic surgical technique. For instance, the acetabular cup of a total hip replacement may be placed at a slightly different angle from normal in order to compensate for a particular patient's pelvic tilt. Alternatively, the replacement knee joint could be altered in its position in a particular way to allow a patient to walk with their normal gait.

These new techniques result in a greater success rate of joint replacement surgeries, but they may take longer or cost a greater amount due to the time necessary to obtain and process the pre-operative information. As such, it is not economically viable to provide all patients with these bespoke techniques. For some patients, the generic surgery is perfectly adequate and causes no problems with the longevity of the joint.

### SUMMARY

It is an object of the present invention to create a method of pre-operatively determining the suitability of a patient's joint for a particular surgical procedure. By examining the patient beforehand, it is possible to distinguish between patients able to be successfully treated using generic surgical techniques and those who require more bespoke surgery. A further object of the invention is to provide a device for successfully determining this suitability.

According to a first aspect of the invention there is provided a joint-orientation monitoring system, preferably for the creation of patient kinematic range data of a joint of a patient, the joint-orientation monitoring system comprising: a joint-orientation monitoring device which determines joint orientation data; and a processor in communication with the joint-orientation monitoring device; the processor determining patient kinematic range data from joint orientation data received from the joint orientation monitoring device.

Patient kinematic range data is useful as it enables joints of patients to be compared to one another and also to standardised average values. This enables simplified monitoring of the anatomy of patients to allow better decision making about surgical procedures.

Preferably, the joint-orientation monitoring system may further comprise a memory element which stores patient kinematic range data and/or preferred kinematic range data.

Additionally, the joint-orientation monitoring system may include a logic element which compares the patient kinematic range data and the preferred kinematic range data.

Comparing the two sets of data allows the difference between the patient kinematic data and the preferred kinematic data to be determined, thus allowing specific decisions about a surgical procedure to be undertaken to be made.

In a preferable arrangement, at least the processor and/or logic element may be parts of a computing device.

Utilising such an arrangement allows commonly available computing devices such as personal computers, tablet computers, or smart phones to be used, lowering the up-front cost of procuring the system.

Optionally, the joint-orientation monitoring device may comprise a wearable portion.

The wearable portion can allow parts of the system to be placed on or close to the patient's body, increasing the accuracy of any measurements taken of the joint.

It may be advantageous for the joint-orientation monitoring device to include a plurality of accelerometers which senses joint motion.

Accelerometers are a relatively cost-effective and simple way of measuring acceleration, but the information harvested by them may be relatively easily translated by a computer into velocity and position information. Therefore, use of accelerometers can provide rapid and accurate positional information to the system of many different points on the body.

It may also be advantageous for the joint-orientation monitoring device to include a video capture device which tracks joint orientation.

Use of a video capture device optionally allows the joint orientation and/or motion of a patient to be recorded without needing to use devices in physical contact with the patient. Avoiding physical contact with the patient ensures that the joint-orientation monitoring device does not hinder the natural movements of the patient, restricting the accuracy of the joint-orientation data. Video capture devices also allow the information to be captured easily and automatically whilst a patient is within the viewable range.

In a preferable arrangement, the video capture device may include depth-sensing means.

Use of depth-sensing means, which allow the video capture device to capture three-dimensional information, allows additional information to be obtained by the video capture device, which improves the accuracy of the overall data gathered.

Optionally, the joint-orientation monitoring device may further include a plurality of markers, trackable by the video capture device, which enhances tracking of joint orientation.

Markers enable the video capture device to more accurately evaluate the relative positions of the patient's anatomy, thus providing more useful information.

It may be advantageous for the joint-orientation monitoring system to further comprise a wireless communication means for wirelessly transferring data between the joint orientation monitoring device and the processor.

By doing so, there is no need for cabling to be provided connecting each component or device. This is especially beneficial when a wearable device is utilised, as it prevents any undesirable limitation of the patient's motion.

According to a second aspect of the invention there is provided a method of pre-operatively determining surgical suitability of a joint, preferably for a surgical corrective procedure, preferably using a joint-orientation monitoring system in accordance with the first aspect of the invention, the method comprising the steps of: a] determining a patient's joint orientation in a plurality of situations; b] determining a patient kinematic range of the patient joint based on the said joint orientations; c] comparing the determined patient kinematic range to a preferred kinematic range; and d] determining that the joint can accept a first surgical corrective procedure if the patient kinematic range falls within the allowable kinematic range, else determining that the joint can accept a second surgical corrective procedure which is different from the said first surgical corrective procedure.

Determining the suitability of a particular patient to receive a specific surgical corrective procedure prevents unnecessary costs being incurred by use of more expensive or time-consuming techniques and procedures where they are not specifically needed.

Beneficially, the patient's joint orientation may be at least partially determined by way of at least one medical imaging technique. These techniques may include at least one of X-ray imaging, computed tomography, or magnetic resonance imaging.

Medical imaging techniques allow joint orientation information to be measured which is not attainable by way of solely external methods or imaging.

In a preferable embodiment, the orientation of the patient's joint may be at least partially determined by way of motion tracking techniques.

Use of motion tracking techniques enables joint orientation information to be obtained without resorting to expensive medical imaging. It also allows patient joint orientation to be viewed in dynamic situations if video, rather than still, imaging is used.

Beneficially, the patient kinematic range is indicative of a range of motion of the patient's joint. Additionally, the preferred kinematic range is indicative of the allowable range of joint motion to qualify for a predetermined surgical corrective procedure.

Typically, the first surgical corrective procedure may be a standard surgical corrective procedure and the second surgical corrective procedure may be an enhanced surgical corrective procedure.

In a preferred embodiment, the first and/or second surgical corrective procedure may be a joint arthroplasty. Whilst other types of surgery may also be suitable, the method is most well suited to joint surgery, including, but not limited to, reconstruction and replacement surgeries.

According to a third aspect of the invention, there is provided a method of pre-operatively determining a surgical suitability of a joint for either a generalised surgical corrective procedure or a bespoke surgical corrective procedure, the method comprising the steps of: a] using a joint-orientation monitoring device, determining patient joint orientation data for a plurality of different kinematic joint positions which are characteristic of activities performed by the patient's joint; b] transmitting the said joint orientation data to a processor; c] computationally determining a patient kinematic range of the patient joint based on the joint orientation data; d] comparing the determined patient kinematic range to a pre-determined preferred kinematic range which is indicative of suitability of the patient joint for the generalised surgical corrective procedure; and e] determining that the joint is suitable for the generalised surgical corrective procedure if the patient kinematic range falls within the preferred kinematic range, else determining that the joint is suitable for the bespoke surgical corrective procedure which is different from the said first surgical corrective procedure.

The invention will now be more particularly described, by way of example only, with reference to the accompanying drawings.

### BRIEF DESCRIPTION OF FIGURES

FIG. 1 shows two pictorial representations of a person, depicting an example of anterior/posterior pelvic tilt during standing and sitting;

FIG. 2 is a flow diagram of a method of pre-operatively determining the suitability of a joint for a surgical corrective procedure, in accordance with the second aspect of the invention.

FIG. 3 is a pictorial representation of a first embodiment of a joint-orientation monitoring system in accordance with the first aspect of the invention;

FIG. 4 is a pictorial representation of a second embodiment of a joint-orientation monitoring system in accordance with the first aspect of the invention;

FIG. 5 is a pictorial representation of a third embodiment of a joint-orientation monitoring system in accordance with the first aspect of the invention; and

### DETAILED DESCRIPTION

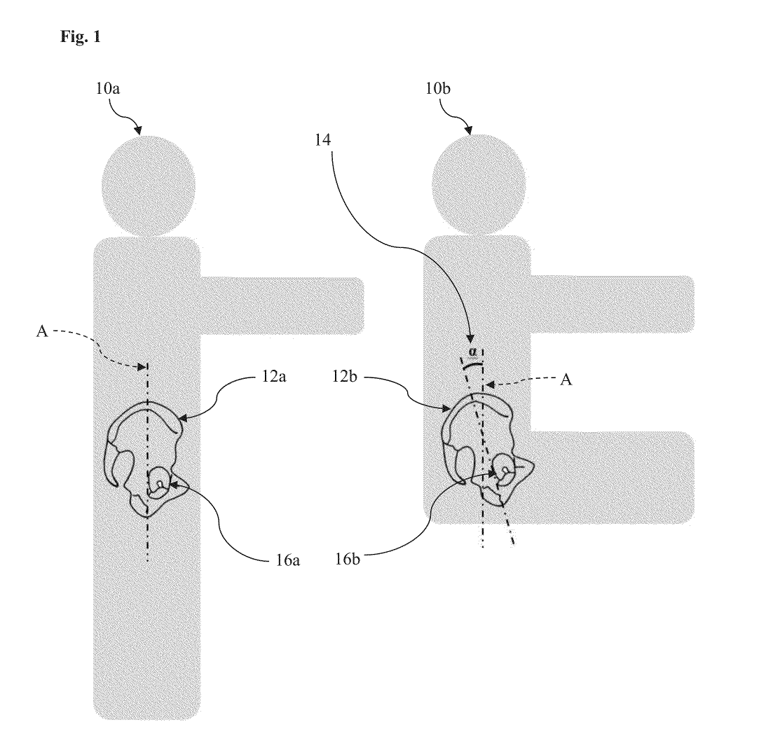

Referring firstly to FIG. 1 of the drawings, there is shown a depiction of a patient 10a, 10b in both standing and seated positions, showing a representation of the pelvic tilt in each. As can be seen from the standing FIG. 10a, the pelvis 12a, in this example, is aligned vertically, indicated by a dotted line A, with no or substantially no anterior or posterior tilt. Conversely, in the seated FIG. 10b, the pelvis 12b presents a posterior pelvic tilt 14 of a degrees. It can be appreciated that this pelvic tilt 14 will result in a different angular position of the femur, which is not shown, relative to the acetabulum 16a, 16b.

This tilt 14, and the resultant relative positioning of the first and second portions of the joint, which in this case are the acetabulum and femur, can be measured in various positions, which are not limited to those depicted in FIG. 1. This relative positioning is hereafter referred to as ‘joint orientation’.

Joint orientation can clearly be exhibited in not only anterior and posterior directions, as shown, but also laterally. The resultant pluralities of joint orientations in three dimensions can be represented by values known as a patient kinematic range, which is preferably indicative of the entire range of the possible joint orientations for a particular joint. For instance, the patient kinematic range could describe the entire range of motion of the femoral head with respect to the acetabular cup. This could be as simple as the maximum angles of motion in posterior/anterior and lateral directions, or as complex as a fully three-dimension map of the movement of the separate parts of the joint.

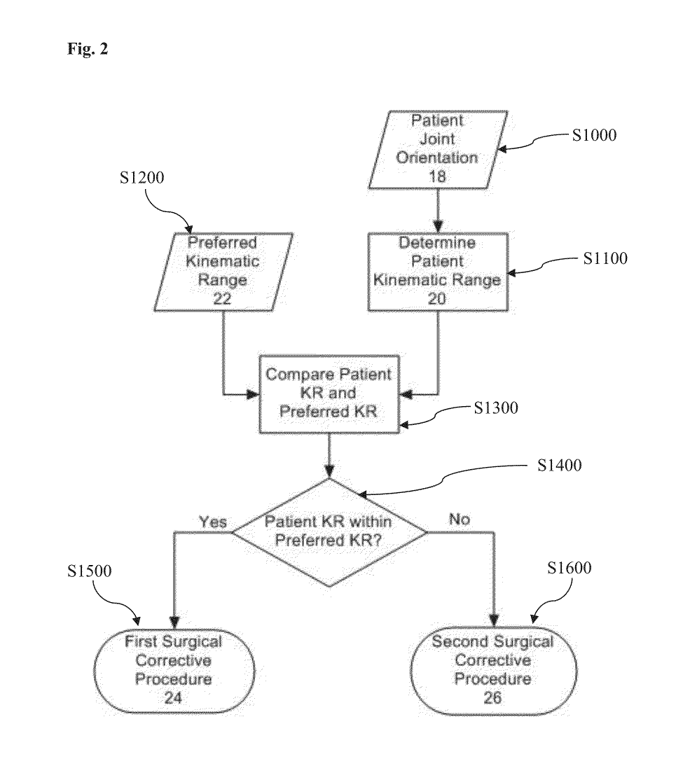

A patient kinematic range for each joint can be utilised in decision-making processes about the treatment plan for each particular joint, as shown in FIG. 2. Measurements of the patient's joint orientation 18 in step S1000 for a particular joint can be converted into a patient kinematic range 20 in step S1100.

The patient kinematic range 20 is specific to each joint of a patient and can impact highly on the success-rate of any particular surgical corrective procedure. Where the patient kinematic range 20 of a joint of a patient is outside of the general range of the population at large, complications can ensue after surgery, as the surgery may be tailored to be most suitable for joints with the kinematic range of the average person, hereafter referred to as a ‘preferred kinematic range’, referenced as 22 and determined at step S1200.

The preferred kinematic range 22 can be produced by comparing the joint orientations of a large number of people to produce an average, preferably modal, range, which is used to create a particular surgical corrective procedure. This information may be procured from patient library data, formed through studies of anatomy. Alternatively, the preferred kinematic range may be determined in a retrospective manner, by studying the method of a surgical corrective procedure to ascertain the joint orientations for which it is most suitably used. Other methods of determining the preferred kinematic range 22 will be obvious to those skilled in the art.

By comparing the patient kinematic range 20 to the preferred kinematic range 22 at step S1300, the suitability of a joint for a particular surgical procedure can be determined. In the present embodiment of the method, if the patient kinematic range is found to be within the preferred kinematic range at step S1400, the patient can be recommended for a first surgical corrective procedure 24 at step S1500. The first surgical corrective procedure 24 will preferably be that which is created to be suitable for the general population, with only the usual level of adjustment available.

Conversely, if the patient kinematic range 20 is found to be outside of the preferred kinematic range 22, the patient may be recommended for a second surgical corrective procedure 26 at step S1600. This second surgical corrective procedure 26 is preferably a bespoke surgery which is capable of sufficiently compensating for the particular anatomy of the patient to provide a higher success-rate than the first surgical corrective procedure 24.

Commonly, whilst generally being an enhanced surgical corrective procedure, the second surgical corrective procedure 26 may be more expensive, time-consuming, or otherwise complicated procedure than the first surgical corrective procedure. As such, it is preferable to utilise the first surgical corrective procedure 24 when a case allows. Therefore, the prescribed method enables the allowability of a particular surgical corrective procedure to be calculated and measured, enabling a surgeon or other decision-maker to provide the best care, when success, cost, and other variables are taken into account.

Whilst the preferred embodiment allows for decisions to be made between two different surgical corrective procedures, it is also foreseeable that the method could be utilised to distinguish a correct or preferred course of action between three or more surgical corrective procedures. For example, a third surgical corrective procedure may be preferable for a joint with a patient kinematic range below the preferred patient kinematic range, or a fourth surgical corrective procedure may be suitable for a joint with a patient kinematic range more than 50% greater than the preferred kinematic range. The list of possibilities hereby disclosed is not intended to be exhaustive and a greater number of iterations of the method of the present invention will be obvious to those skilled in the art.

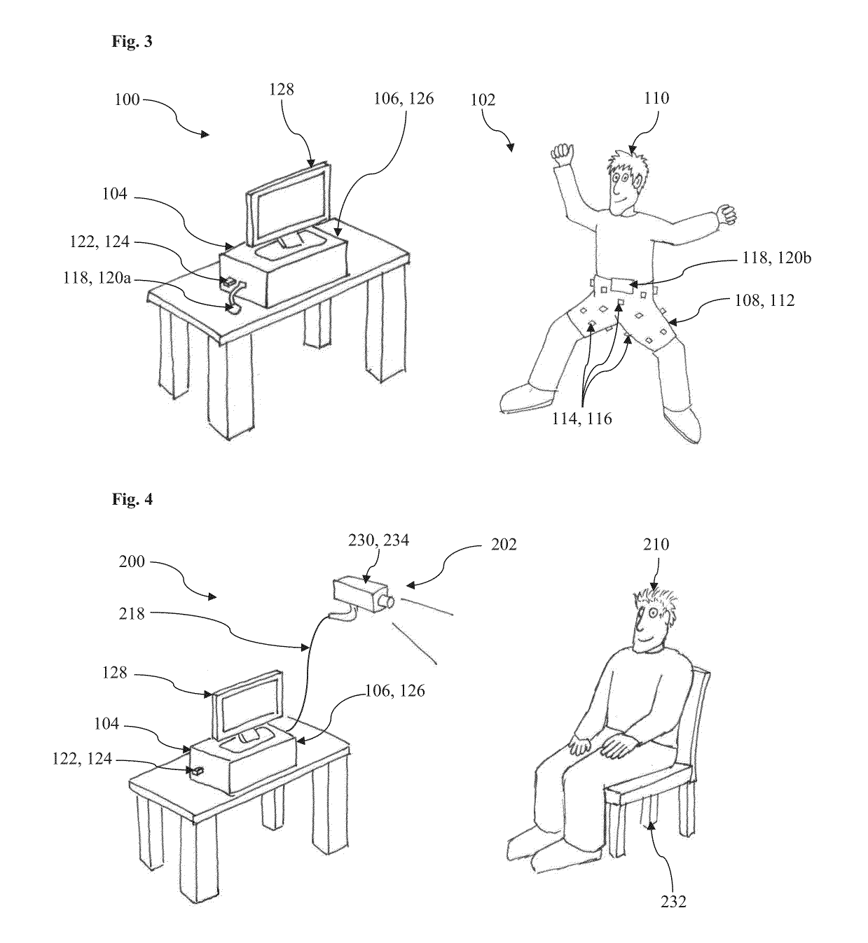

Three embodiments of a system for the creation of patient kinematic range data are depicted in FIGS. 3 to 5. The embodiment of FIG. 3, indicated globally at 100, comprises a joint-orientation monitoring device 102 and a personal computer 104, having at least a processor 106.

The joint-orientation monitoring device 102 may typically include a wearable device 108 worn by a patient 110, which in this embodiment is a pelvic garment 112, embedded with a plurality of sensors 114. The sensors are more particularly accelerometers 116, which are therefore suited to detecting acceleration of a number of different points on the pelvic garment 112 and therefore pelvis and femur. As the pelvic garment 112 is preferably tight-fitting or snug, the accelerometers 116 each detect the acceleration of a point on the patient's body 110, which is hereby referred to as ‘joint orientation data’.

Evidently, a pelvic garment 112 is suitable for the detection of the pelvic orientation, and similar joint-orientation monitoring devices can be imagined for other joints.

The joint orientation data is relayed from the pelvic garment 112 to the computer 104 via, preferably wireless, communication means 118. This wireless communication means 118 comprises a first transponder unit 120a on the pelvic garment 112 and a second transponder unit 120b in communication with the computer 104.

The first and second transponder units 120a, 120b communicate via radio waves in this embodiment, but it is equally plausible that they may instead communicate by microwaves, infrared radiation, Bluetooth®, or any other wireless communication method or suitable data transmission protocol. It would also be possible for the pelvic garment 112 and computer 104 to be interconnected in a wired fashion, but this may be disadvantageous due to the dangers of trailing wires and the undesired tethering of the patient 110, which could affect joint orientation data.

The received joint-orientation data is processed by the processor 106, housed within the computer 104. Joint-orientation data, which in this case is received as recorded acceleration data, can be translated by the processor 106 to indicate the relative positions of each data-providing accelerometer 116 and therefore the related accelerations of various pelvic positions. As such, patient kinematic range data can be produced, which in this case is indicative of the joint orientation of the patient's hip joints.

The computer 104 further includes a memory element 122, in this case for example a USB flash drive 124, upon which is stored preferred kinematic range data. A logic element 126, used to compare the preferred kinematic range data with the patient kinematic range data, is included within the computer 104, and in this case is contained within the processor 106. The joint-orientation monitoring system can therefore also determine the relationship between the patient kinematic range data and the preferred kinematic range data, which can then be displayed on a monitor 128 of the computer 104, if required.

Whilst shown as a USB flash drive 124, the memory element 122 may additionally or alternatively be provided by way of a hard disk drive, solid state drive, or other memory type. Similarly, data output, which is provided by the monitor 128, may additionally or alternatively be provided by other data output means, such as a speaker or printer, or transmitted electronically, either in a wired or wireless manner.

FIG. 4 depicts a second embodiment of a joint-orientation monitoring system. Similar or identical features have been omitted from further description, for brevity.

The joint-orientation monitoring device 202 of the second embodiment, indicated globally as 200, is a video capture device 230, connected to the computer 104 via a wired communication means 218. The patient 210 of this embodiment is in a seated position on a chair 232, and the video capture device 230 is able to determine visually the orientation of the patient's joints. Enhanced joint orientation data can be captured through the use of depth-sensing means 234, which are also provided by the video capture device 230. Depth-sensing means 234 are provided by embedded infrared emission and detection within the video capture device 230.

The video capture device 230 is therefore able to track the motions and positions of any joints of the patient 210, as long as the patient 210 is within a viewable field of the video capture device 230. Advantages of the use of the video capture device 230 include that the patient 210 may not be required to wear restricting devices such as the pelvic garment 112 of the first embodiment 100. However, the wearing of such tight garments may advantageously allow the video capture device 230 to make more accurate determinations of joint orientation data.

The processor 106 is able to manipulate the joint orientation data, received from the video capture device 230 as image and depth data, and translate this into the required patient kinematic range data. The video capture device 230, whilst described as being capable of capturing joint motion, may also be capable of taking still images, with or without integrated depth data, which can also be used to determine the patient kinematic range data.

A video capture device 230 may be used in conjunction with a pelvic garment similar to that of the first embodiment 100 in order to provide a different manner of joint-orientation detection. For instance, by replacing the accelerometers 116 of the first embodiment 100 with a plurality of markers, for example infrared-reflective markers, a video capture device 230 may be used in place of the accelerometers 116 for detection of the relative positions of the markers. This technique is used in the film industry for motion-capture of the human body, and therefore similar joint-orientation monitoring devices may be incorporated into the joint-orientation monitoring system of the present invention.

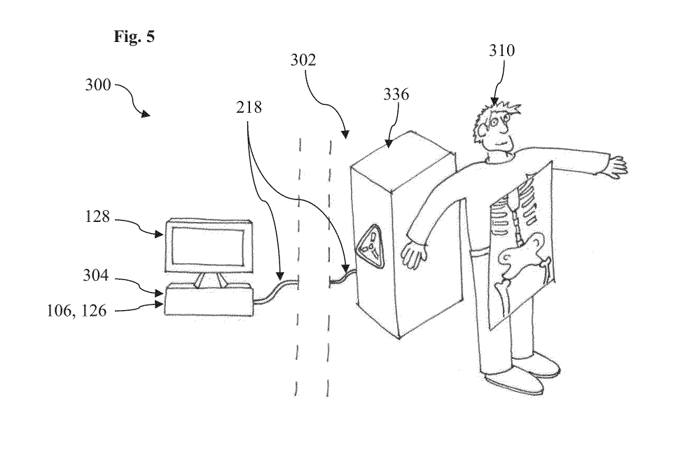

A third embodiment of the joint-orientation monitoring system, depicted in FIG. 5 and indicated globally as 300, utilises a computer 304 which is remote from the joint-orientation monitoring device 302. This allows the computer 304 to be operated by an individual remote to a patient 310, which may be advantageous. Again, similar references refer to parts which are similar or identical to those of the preceding embodiments, and further detailed description is therefore omitted.

The joint-orientation monitoring system 300 of the third embodiment is limited to the taking of still images, due to the use of an X-ray scanner 336 as the joint-orientation monitoring device 302. However, by the use of the X-ray scanner 336, the joint-orientation monitoring system 300 may provide more accurate joint orientation data to the computer 304 as the joint itself can be directly imaged. This may be particularly useful in cases where the patient 310 is particularly overweight or obese, where the joints may be hidden beneath a thick layer of adipose tissue. This layer could cloak the joints from other types of joint-orientation monitoring device, making the system less useful.

The more accurate joint-orientation data may be utilised to provide more accurate patient kinematic range data, which can therefore be more useful in a decision-making process.

Whilst an X-ray scanner 336 has been utilised in this embodiment, other medical imaging techniques may be utilised, dependent on choice. For instance, computed tomography can be used to generate 3D models of patient joint anatomy, which can again enhance the accuracy of the patient kinematic range data, or alternatively a magnetic resonance imaging system may be used, if X-rays are not desirable for reasons such as excessive exposure to radiation.

The joint-orientation monitoring system 100; 200; 300 should be used to monitor a plurality of different joint positions or motions in order to provide the patient kinematic range data which is most characteristic of the patient's joint. These positions are dependent on the joint for which the patient kinematic range data is being determined. For instance, if a hip joint is being analysed, it may be preferable to view the patient whilst sitting, standing, performing squats, running, and/or other activities which provide a good range of pelvic movement. Similarly, if a knee is to be analysed, similar activities may be analysed. However, if a shoulder is being analysed, it may be more useful to view throwing, arm, swinging, and/or elevation of the shoulder joint. Particular activities will be obvious to the skilled person which are particularly useful for whichever joint is being analysed.

Additionally, whilst the embodiments depicted in FIGS. 3 to 5 show joint-orientation monitoring systems monitoring the orientation of the patient's hip joint, the systems are equally well suited to monitoring of the knee, shoulder, ankle, or any other anatomical joint. The systems depicted, or further embodiments of such systems, may be used individually or may preferably be used in tandem with one another, such that the most complete joint-orientation data may be provided and therefore the most accurate patient kinematic data may be determined.

It is therefore possible to provide a joint-orientation monitoring system, for creation of patient kinematic range data from a plurality of joint orientations, along with a method for comparing this patient kinematic range data to preferred kinematic range data in order that an educated decision can be made between first and second surgical corrective procedures. The device and method allow enhanced decision-making to be performed, ensuring the patient is submitted for the most optimal surgical corrective procedure.

The words ‘comprises/comprising’ and the words ‘having/including’ when used herein with reference to the present invention are used to specify the presence of stated features, integers, steps or components, but do not preclude the presence or addition of one or more other features, integers, steps, components or groups thereof.

It is appreciated that certain features of the invention, which are, for clarity, described in the context of separate embodiments, may also be provided in combination in a single embodiment. Conversely, various features of the invention which are, for brevity, described in the context of a single embodiment, may also be provided separately or in any suitable sub-combination.

The embodiments described above are provided by way of examples only, and various other modifications will be apparent to persons skilled in the field without departing from the scope of the invention herein described and defined.

What is claimed is:

1. A joint-orientation monitoring system comprising: a joint-orientation monitoring device which determines joint orientation data of the patient; and a processor in communication with the joint-orientation monitoring device; the processor determining patient kinematic range data from the joint orientation data received from the joint orientation monitoring device.

2. The joint-orientation monitoring system as claimed in claim 1, further comprising a memory element which stores patient kinematic range data and/or preferred kinematic range data.

3. The joint-orientation monitoring system as claimed claim 2, including a logic element which compares the patient kinematic range data and the preferred kinematic range data.

4. The joint-orientation monitoring system as claimed in claim 2, wherein at least the processor and/or logic element are parts of a computing device.

5. The joint-orientation monitoring system as claimed in claim 4, wherein the joint orientation monitoring device comprises a wearable portion.

6. The joint-orientation monitoring system as claimed in claim 5, wherein the joint-orientation monitoring device includes a plurality of accelerometers which sense joint motion.

7. The joint-orientation monitoring system as claimed in claim 6, wherein the joint-orientation monitoring device includes a video capture device which tracks joint orientation.

8. The joint-orientation monitoring system as claimed in claim 7, wherein the video capture device includes depth-sensing means.

9. The joint-orientation monitoring system as claimed in claim 6, including a plurality of markers, trackable by the video capture device, which enhances tracking of joint orientation.

10. The joint-orientation monitoring system as claimed in claim 1, further comprising a wireless communicator which wirelessly transfers data between the joint orientation monitoring device and the processor.

11. A method of pre-operatively determining surgical suitability of a joint , the method comprising the steps of:
a] determining a patient's joint orientation in a plurality of situations;
b] determining a patient kinematic range of the patient joint based on the said joint orientations;
c] comparing the determined patient kinematic range to a preferred kinematic range; and
d] determining that the joint can accept a first surgical corrective procedure if the patient kinematic range falls within the preferred kinematic range, else determining that the joint can accept a second surgical corrective procedure which is different from the said first surgical corrective procedure.

12. The method as claimed in claim 11, wherein the orientation of the patient's joint is at least partially determined by way of at least one medical imaging technique.

13. The method as claimed in claim 12, wherein the said at least one medical imaging technique includes at least one of X-ray imaging, computed tomography, or magnetic resonance imaging.

14. The method as claimed in claim 11, wherein the patient's joint orientation is at least partially determined by way of motion tracking techniques.

15. The method as claimed in claim 11, wherein the patient kinematic range is indicative of a range of motion of the patient's joint.

16. The method as claimed in claim 11, wherein the preferred kinematic range is indicative of the allowable range of joint motion to qualify for a predetermined surgical corrective procedure.

17. The method as claimed in claim 11, wherein the first surgical corrective procedure is a standard surgical corrective procedure.

18. The method as claimed in claim 11, wherein the second surgical corrective procedure is an enhanced surgical corrective procedure.

19. The method as claimed in claim 11, wherein the first and/or second surgical corrective procedure is a joint arthroplasty.

20. A method of pre-operatively determining a surgical suitability of a joint for either a generalised surgical corrective procedure or a bespoke surgical corrective procedure, the method comprising the steps of:
a] using a joint-orientation monitoring device, determining patient joint orientation data corresponding to a plurality of different kinematic joint positions which are characteristic of activities performed by the patient's joint;
b] transmitting the said joint orientation data to a processor;
c] computationally determining a patient kinematic range of the patient joint based on the joint orientation data;
d] comparing the determined patient kinematic range to a predetermined preferred kinematic range which is indicative of suitability of the patient joint corresponding to the generalised surgical corrective procedure; and
e] determining that the joint is suitable for the generalised surgical corrective procedure if the patient kinematic range falls within the preferred kinematic range, else determining that the joint can accept the bespoke surgical corrective procedure which is different from the said first surgical corrective procedure.
